(19) United States
(12) Patent Application Publication
(10) Pub. No.: US 2020/0197685 A1
(43) Pub. Date: Jun. 25, 2020

(54) RELEASABLE CONNECTION ASSEMBLY

(71) Applicant: NORDSON CORPORATION, WESTLAKE, OH (US)

(72) Inventors: Kyle R. STEELE, Windsor, CO (US); Peter D. Lewis, Loveland, CO (US)

(21) Appl. No.: 16/608,808

(22) PCT Filed: Apr. 27, 2018
(86) PCT No.: PCT/US2018/029724

§ 371 Date: Oct. 25, 2019

Related U.S. Application Data
(60) Provisional application No. 62/490,716, filed on Apr. 27, 2017.

(51) Int. Cl. A61M 39/10; F16L 37/084; F16L 33/30

(52) U.S. Cl. CPC A61M 39/1011; A61M 39/105; A61M 2039/1027; F16L 33/30; A61M 2039/1016; F16L 37/0847

(57) ABSTRACT

A releasable connection assembly for connecting tubing sections is disclosed. The assembly includes a male bayonet connector including a shaft including a distal end portion comprising a lumen extending through the shaft, and a sealing surface configured to create a fluid-tight seal between the lumen and a female receiving lumen. The male bayonet connector also includes a latch member positioned adjacent to the shaft for coupling to a female latch structure. The female receiving member has a proximal end that defines at least two openings configured to receive at least part of the distal end portion of the shaft and the latch member.

### CROSS REFERENCE TO RELATED APPLICATIONS

This application claims the benefit of U.S. Provisional Patent App. No. 62/490,716, filed Apr. 27, 2017, the disclosure of which is hereby incorporated by reference herein.

### TECHNICAL FIELD

The present disclosure relates generally to the field of medical devices used for the transport of both gaseous and liquid fluids and more specifically, to a male bayonet connector and a female latch member for creating a releasable air and fluid seal connection between one or more sections of tubing.

### BACKGROUND

Tubing sections, for example, medical tubing, must often be joined together to provide for gas and/or liquid fluid flow from one component to another. It is further often desirable to connect and disconnect tubing sections from one another. For example, when a patient's blood pressure is taken with an automatic blood pressure monitor, tubing from the blood pressure cuff (which is generally wrapped around the patient's arm) is connected to the tubing that is connected to the blood pressure monitor. To disconnect the cuff from the blood pressure monitor, it is desirable to detach the tubing section connected to the cuff from the tubing connected to the blood pressure monitor. Similarly, when providing intravenous fluids, it is often required to replace an empty fluid bag with a full fluid bag without removing the intravenous needle or stent from the patient. In order to switch between the first fluid bag and the second fluid bag, it is desirable to detach a tubing section connected with the fluid bag to the tubing section connected with the needle or stent placed intravenously in the patient, which can then be easily connected with a tubing section connected with the new fluid bag.

Existing tubing connectors are prone to leakage and unwanted disconnection when the patient is still receiving treatment via the connected tubes due to side-loads caused by the weight of the connected tubes and components, as well as accidental pulling of the tubes by the patient or medical personnel.

Furthermore, certain medical devices require the use of multiple tubes for supplying air or other fluid between the patient and the device. For example, certain models of blood pressure monitors, such as the Dinamap Procare series, manufactured by General Electric, employ dual tubes for connecting the blood pressure cuff to the monitor. As such, a connector including multiple fluid passages for directing airflow between the tube segments is desirable, so as to avoid having to individually connect and disconnect multiple connectors when hooking or unhooking a patient to the monitor.

Furthermore, a new standard for tubing connectors, ANSI/AAMI/ISO 80369-5:201x, applies to connectors and specifies allowable geometry limits, among other limitations. An objective of the standard is to eliminate accidental misconnects. As such, a connector that complies with the new standard and solves the limitations of prior art connectors is desirable.

### SUMMARY

An improved latching mechanism and an alternate method of connection is desired so as to minimize the risk of premature disconnect and to maintain fluid-tight seals during use. A dual male bayonet connector is disclosed that includes a center post between two shafts that latches on a feature independent from the features of the two shafts. A spring loaded button is used to connect and to disconnect the center post to a female latch connector; however, as described in further detail below, alternate methods of latching are possible.

The center post between the two shafts can be located so there is symmetry to where it doesn't matter which orientation the connection between the male bayonet connector and the female latch connector is made. The center post may also include a lead geometry to activate and depress a spring actuated latching mechanism within the female latch connector.

In known designs, a fluid lumen is utilized as a location for the latching mechanism. However, by using a retention post (also referred to as a center post or latch member), the latch mechanism does not need to contact the sealing areas of the lumen or be in a location behind the seal which significantly increases complexity. In addition, the tolerance stack-ups are easier to maintain since there can be only one latching feature. Yet further, tool maintenance costs are reduced since there are fewer critical shutoffs. In the disclosed designs, the flow lumen can have more uniform wall section which improves manufacturability and multiple lumen sizes and shaft sizes can be used without affecting the latching mechanism. Alternatively, the central latching post may be offset to allow for various connection orientations, for example, where only one flow orientation is desired.

In a preferred aspect, the latch member is located in a central location between the dual lumens to allow for uniform loading of the latch mechanism. The centrally located latch member is inserted into a slot between the flow paths defined in the female latch connector and contacts the latching mechanism, such as a spring actuated slide latch or other mechanism. The latching mechanism may be positioned within an enclosure of the female latch connector. In the case of a sliding spring latch mechanism, an angular lead geometry of the retention post would push the latching mechanism aside until a locking groove or notch formed in the retention post allows the latching mechanism to move into the groove which retains the latch member within the female latch connector.

Clearance at the top of the latching slot allows for the latching mechanism to move aside and not interfere with the retention post. This is important to allow the latching mechanism to move aside and then move back into the original location, where the groove and latching mechanism hold the male and female connectors together.

If grooves are present on the centrally located retention post, they would be preferably symmetrically located to ensure proper function when the connection is made in alternate orientations. For example, if the male bayonet connector is inserted into the female latch connector in an upside down orientation.

The latch member may include various cross sectional shapes, for example, circular, rectangular, or still other shapes.

A male bayonet connector is disclosed. The male bayonet connector includes a first shaft, a second shaft, and a latch member positioned between the first and second shafts. The first shaft includes a first distal end portion that comprises a first lumen extending through the first shaft, and a first sealing surface configured to engage a female receiving member to create a fluid-tight seal between the first lumen and a first female receiving lumen. The second shaft includes a second distal end portion comprising a second lumen extending through the second shaft, and a second sealing surface configured to engage the female receiving member to create a fluid-tight seal between the second lumen and a second female receiving lumen. The latch member configured to couple to the female receiving member.

A male bayonet connector can also include a shaft including a distal end portion and a lumen extending through the shaft. The shaft also includes a sealing surface configured to create a fluid-tight seal between the lumen and a female receiving lumen. The male bayonet connector also includes a latch member positioned adjacent to the shaft and configured to couple to a female latch structure. A releasable connection assembly including the male bayonet connector and a female receiving member is also disclosed.

A releasable connection assembly is also disclosed. The releasable connection assembly includes a male bayonet connector and a female receiving member. The male bayonet connector includes a first shaft including a first distal end portion having a first sealing surface and a first lumen extending therethrough, and a second shaft including a second distal end portion having a second sealing surface and a second lumen extending therethrough. The male bayonet connector also includes a latch member positioned between the first and second shafts. The female receiving member includes a proximal end defining a first receiving lumen and a second receiving lumen configured to receive the first shaft and the second shaft within, respectively, such that a fluid-tight seal is formed between both the first shaft and the first receiving lumen, and the second shaft and the second receiving lumen. The proximal end also defines a latch receiving lumen configured to receive the latch member within, such that such that motion between the male bayonet connector and the female receiving member is substantially prevented.

This summary is not intended to identify key features or essential features of the claimed subject matter, nor is it intended to be used to limit the scope of the claimed subject matter. A more extensive presentation of features, details, utilities, and advantages of the present invention is provided in the following written description of various embodiments of the invention, illustrated in the accompanying drawings, and defined in the appended claims.

### BRIEF DESCRIPTION OF THE DRAWINGS

FIG. 1 is a front isometric view of a male dual bayonet connector, a female latch connector, and tubing sections.

FIG. 2 is a rear isometric view of the male dual bayonet connector shown in FIG. 1.

FIG. 3 is a front elevation view of the male dual bayonet connector shown in FIG. 1.

FIG. 4 is a top plan view of the male dual bayonet connector shown in FIG. 1.

FIG. 5 is a rear elevation view of the male dual bayonet connector shown in FIG. 1.

FIG. 6 is a side elevation view of the male dual bayonet connector shown in FIG. 1.

FIG. 7 is a rear isometric view of the male dual bayonet connector and female latch connector in cross section of the connecting member taken along line 7-7 of FIG. 1.

FIG. 8 is an exploded rear isometric view of the female latch connector shown in FIG. 1.

FIG. 9 is a front isometric view of a latch structure of the female latch connector.

FIGS. 10-12 are side cross sectional views of a latch member positioned within a female latch connector.

FIG. 13 is a side cross sectional view of the male bayonet connector positioned within the female latch connector.

FIGS. 14A and 14B illustrate an expanded front isometric view of an alternative aspect of a male dual bayonet connector and a female latch connector.

FIGS. 15A and 15B illustrate an expanded front isometric view of another alternative aspect of a male dual bayonet connector and a female latch connector.

FIGS. 16A and 16B illustrate an expanded front isometric view of another alternative aspect of a male dual bayonet connector and a female latch connector.

FIG. 17 illustrates a front isometric view of an alternative aspect of a male dual bayonet connector positioned within a female latch connector.

### DETAILED DESCRIPTION

Male bayonet connectors, in conjunction with female latch connectors, may be used to releasably connect sections of tubing. In one embodiment, the male bayonet connector may have a single shaft portion defining a single lumen therethrough and an outer sealing surface that is configured to engage an inner surface of a female latch connector to form a gas and/or liquid fluid seal between the male and female components. The female latch connector may include a latching mechanism that engages a latch member of the male bayonet connector so as to prevent removal of the male bayonet connector when connected with the female connector. In another embodiment, the male bayonet connector may have dual shafts, each defining a lumen therethrough. In alternative embodiments, the male bayonet connector may have three or more shafts defining three or more lumens. In embodiments of multiple lumen male bayonet connectors, a grip portion may be used to join the shaft portions, as well as tubing couplings that are configured to engage and retain multiple sections of tubing.

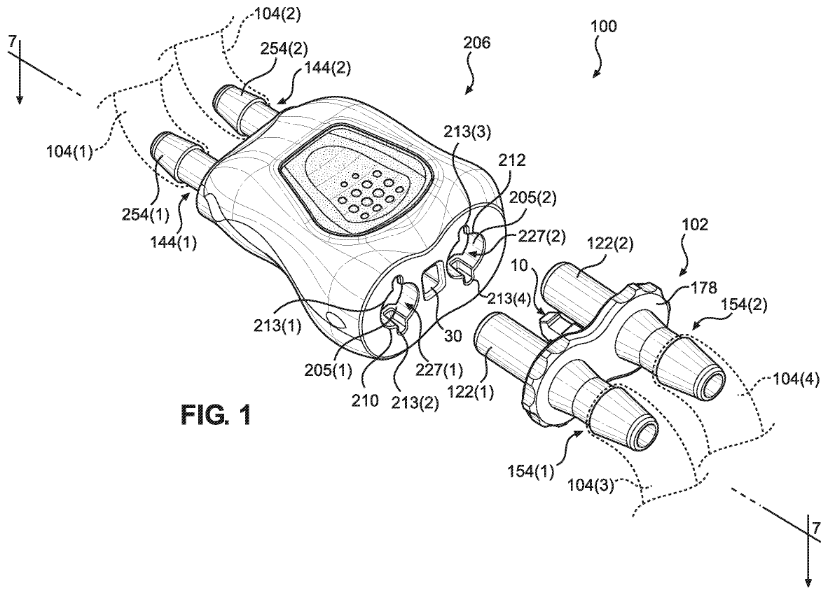

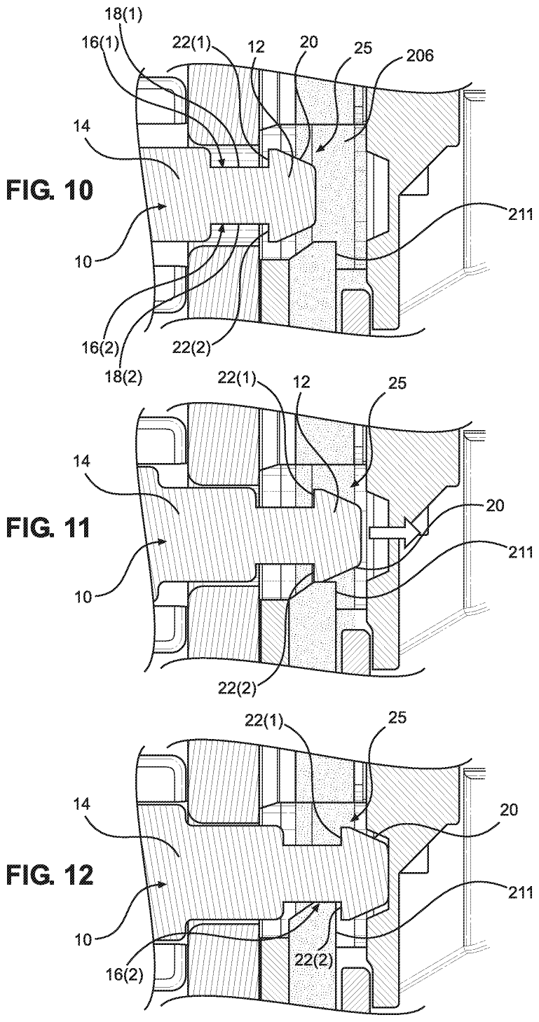

An exemplary environment for a male dual bayonet connector 102 is illustrated in FIG. 1. The environment may include a releasable connection assembly 100 and tubing sections 104(1)-104(4). The releasable connection assembly 100 may include the male dual bayonet connector 102 and a female latch connector 206. The male dual bayonet connector 102 may be connected with the female latch connector 206 as will be described further with respect to FIGS. 10-12.

Referring to FIG. 1, first and second tubing sections 104(1)-104(2) may connect with respective tubing couplings 144(1)-144(2) on the distal end of the female latch connector 206. Third and fourth tubing sections 104(3)-104(4) may connect with respective tubing couplings 154(1)-154(2) on the proximal end of the male dual bayonet connector 102. As will be described further below, the male dual bayonet connector 102 may be connected with the female latch connector 206 by inserting the distal end of the male dual bayonet connector 102 into receiving openings 205(1)-205(2) and latching receiving opening 30 defined in the proximal end of the female latch connector 206. The orientations “proximal” and “distal” as used herein have been arbitrarily chosen, and are not meant to limit the present disclosure, but will follow the convention just described with reference to the ends of the female latch connector 206 and male dual bayonet connector 102.

Certain terminology is used in the description for convenience only and is not limiting. The words “top”, “bottom”, “above,” and “below” designate directions in the drawings to which reference is made. The term “substantially” is intended to mean considerable in extent or largely but not necessarily wholly that which is specified. The terminology includes the above-listed words, derivatives thereof and words of similar import.

The first exterior opening 205(1) may include a first perimeter 210, a first slot 213(1), and a second slot 213(2). The first perimeter 210 may extend circumferentially about a first center of the first exterior opening 205(1). The first slot 213(1) and the second slot 213(2) may extend radially outward from the first perimeter 210. In an aspect, the first slot 213(1) may be spaced 180 degrees from the second slot 213(2) about the first perimeter 210.

The second exterior opening 205(2) may include a second perimeter 212, a third slot 213(3), and a fourth slot 213(4). The second perimeter 212 may extend circumferentially about a second center of the second exterior opening 205(2). The third slot 213(3) and the fourth slot 213(4) may extend radially outward from the second perimeter 212. In an aspect, the third slot 213(3) may be spaced 180 degrees from the fourth slot 213(4) about the second perimeter 212.

In alternative aspects, each of the first and second exterior openings 205(1)-205(2) may include fewer or more slots extending from their respective first and second perimeters 210 and 212. Each of the slots may be spaced at various distances from each other slot along their respective perimeter.

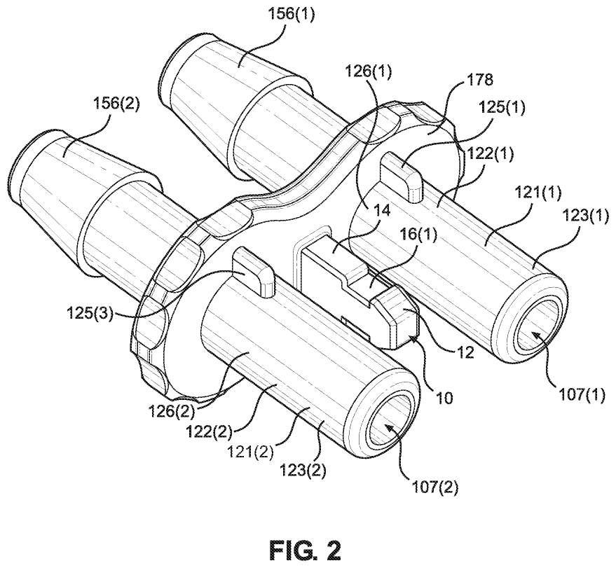

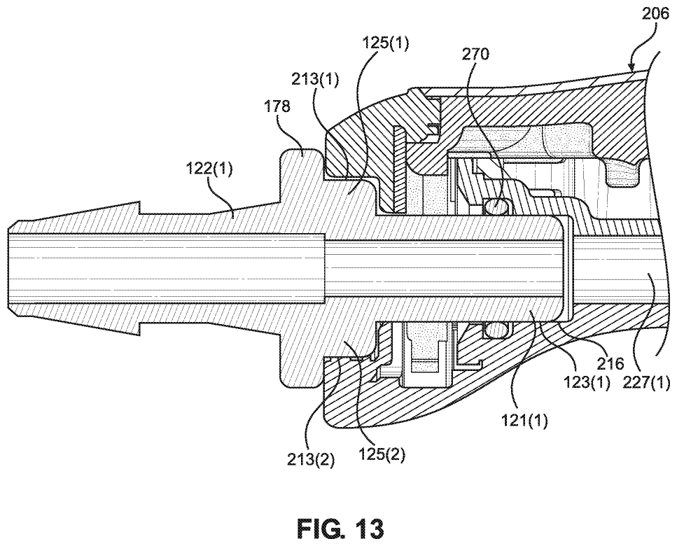

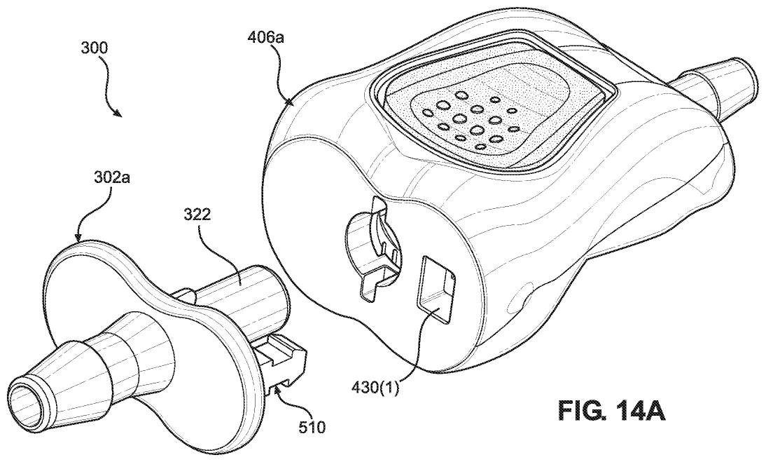

The male dual bayonet connector 102 is illustrated in greater detail in FIGS. 2-6. The male dual bayonet connector 102 may include dual shafts 122(1)-122(2) connected by a grip 178. The dual shafts 122(1)-122(2) may extend from the proximal end of the male bayonet connector 102 to the distal end of the connector 102. As best seen in FIGS. 2, 3 and 5, each of the dual shafts 122(1)-122(2) may define a cylindrical lumen 107(1)-107(2) for transporting fluid from the third and fourth tubing sections 104(3)-104(4) coupled with the male dual bayonet connector 102 to the first and second tubing sections 104(1)-104(2) coupled with the female latch connector 206 via corresponding cylindrical lumens 227(1)-227(2) defined in the female latch connector 206. The dual cylindrical lumens 107(1)-107(2) of the shafts 122(1)-122(2) may be of substantially uniform diameter throughout the entire length of the dual shafts 122(1)-122(2) or, as best shown in cross-section in FIG. 13, may narrow or widen in diameter along the length of the shafts 122(1)-122(2) to include a smaller diameter section and a larger diameter section. In other embodiments, the diameter of the dual cylindrical lumens 107(1)-107(2) may be constant. In further embodiments, the portion of lumens 107(1)-107(2) in the tubing couplings 144(1)-144(2) may be radially offset with respect to the portion of the lumens 107(1)-107(2) along the length of the shafts 122(1)-122(2) to accommodate different tubing configurations. For example, the diameters of the dual cylindrical lumens 107(1)-107(2) may be larger and/or the tubing couplings 144(1)-144(2) may be spaced further or closer apart than the dual shafts 122(1)-122(2) to accommodate varying thicknesses of walls of tubing 104(1)-104(2).

The dual lumen configuration of the male dual bayonet connector 102 allows for simultaneously connecting and disconnecting two or more tubes using a single connection assembly, rather than requiring a separate connection assembly for each tube. As such, the male bayonet connector 102 may provide more efficient connecting and disconnecting of tubes by reducing the amount of time required for medical personnel to hook and unhook a patient from medical equipment.

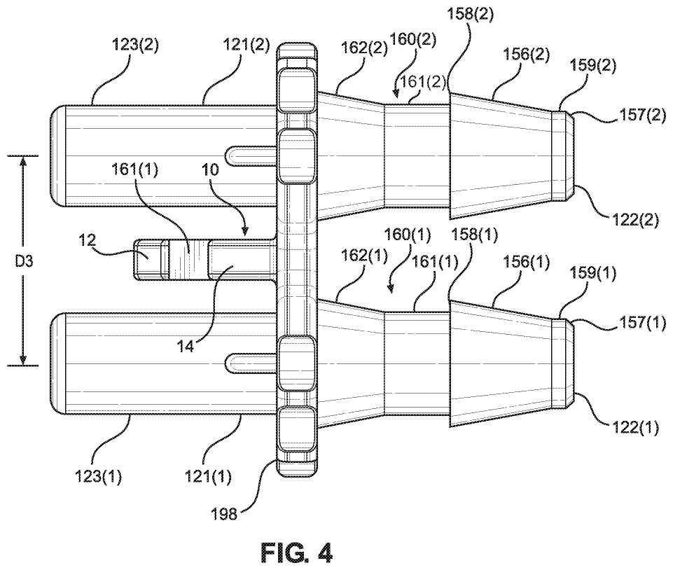

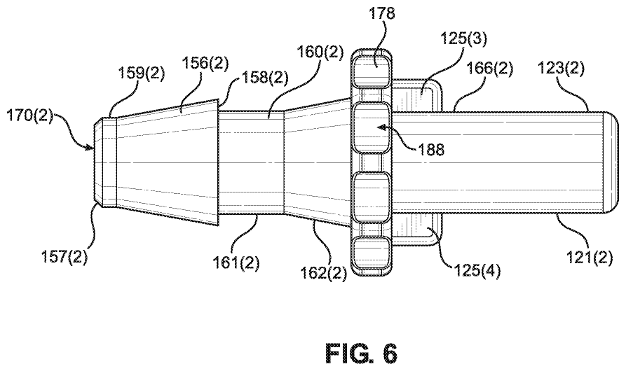

The proximal ends of the dual shafts 122(1)-122(2) may each include a coupling end 156(1)-156(2) shaped as a frustum tapering toward the proximal end for coupling with the third and fourth tube sections 104(3)-104(4) (as seen in FIG. 1). As best seen in FIGS. 4 and 6, the coupling ends 156(1)-156(2) may include a flattened region 159(1)-159(2) toward the proximal ends of the coupling ends 156(1)-156(2), i.e., at the smaller diameter of the frustum. The proximal ends of the coupling ends 156(1)-156(2) may further define a chamfered edge 157(1)-157(2). The exact angle of the chamfered edge 157(1)-157(2) may vary. For example, the chamfered edge 157(1)-157(2) may be between 30-55 degrees. In other embodiments, the proximal ends of the coupling ends 156(1)-156(2) may be rounded or perpendicular to the flattened region 159(1)-159(2). The overall tapered configuration of the coupling ends 156(1)-156(2), including the flattened regions 159(1)-159(2) and chamfered edges 157(1)-157(2) of the coupling ends 156(1)-156(2), may facilitate the insertion of the third and fourth tubing sections 104(3)-104(4) over the coupling ends 156(1)-156(2) of the dual shafts 122(1)-122(2).

The distal ends of the coupling ends 156(1)-156(2), i.e., at the larger diameter of the frustum, may be adjacent to a coupling shaft portion 160(1)-160(2) that may have a first portion 161(1)-161(2) having a narrower outer diameter than that of the distal end of the coupling end 156(1)-156(2), as well as a second portion 162(1)-162(2) that gradually widens in outer diameter toward the grip 178. As such, the coupling shaft portions 160(1)-160(2) may, in some embodiments, vary in outer diameter along the length of the shafts 122(1)-122(2), but in other embodiments, may have a substantially uniform outer diameter that may be narrower than the distal end of the coupling end 156(1)-156(2). The difference in outer diameters between the coupling ends 156(1)-156(2) and the first portions 161(1)-161(2) of the coupling shaft portions 160(1)-160(2) may result in an annular shelf that functions as a coupling barb 158(1)-158(2) for retaining the third and fourth tubing couplings 104(3)-104(4).

The distal ends of the shafts 122(1)-122(2) may define a sealing portion 121(1)-121(2) including a flattened sealing surface 123(1)-123(2). As will be described in further detail below, each sealing surface 123(1)-123(2) may engage a respective sealing member 270 (as shown in, e.g., FIG. 13) in the female latch connector 206 to create a fluid-tight seal between the male dual bayonet connector 102 and the female latch connector 206. The distal end of the sealing portion 121(1)-121(2) may be rounded, as shown in FIGS. 2, 4 and 6, or, in other embodiments, may be chamfered or perpendicular to the sealing surface 123(1)-123(2).

The distal end of the dual shafts 122(1)-122(2) may include ribs 125(1)-125(4) and rib surfaces 126(1)-126(2). The rib surfaces 126(1)-126(2) are proximal to the sealing surfaces 123(1)-123(2). The rib surfaces 126(1)-126(2) extend from the grip 178 to the sealing surfaces 123(1)-123(2). Each of the rib surfaces 126(1)-126(2) includes a pair of ribs 125(1)-125(4) that extend radially outward. For example, rib surface 126(1) includes the ribs 125(1)-125(2). Each of the ribs 125(1)-125(2) extend radially from the rib surface 126(1). In an aspect of this disclosure, each of the ribs 125(1)-125(2) extend in radially opposite directions from the rib surface 126(1), and each of the ribs 125(1)-125(2) may be coupled to the grip 178. In a similar manner, the rib surface 126(2) includes the ribs 125(3)-125(4). Each of the ribs 125(3)-125(4) extend radially from the rib surface 126(2). In an aspect of this disclosure, each of the ribs 125(3)-125(4) extend in radially opposite directions from the rib surface 126(2), and each of the ribs 125(3)-125(4) may be coupled to the grip 178.

FIG. 13 is a cross sectional view illustrating the male dual bayonet connector 102 positioned within the female latch connector 206. The ribs 125(1)-125(2) of the male dual bayonet connector 102 and the slots 213(1)-213(2) of the female latch connector 206 are configured such that the ribs 125(1)-125(2) may fit within the slots 213(1)-213(2) when the male dual bayonet connector 102 is coupled to the female latch connector 206. Similarly, ribs 125(3)-125(4) and the slots 213(3)-213(4) are configured such that the ribs 125(3)-125(4) may fit within the slots 213(3)-213(4). The coupling of the ribs 125(1)-125(4) within the slots 213(1)-213(4) may provide added support to the sealing surfaces 123(1)-123(2), which may prevent the seal between male dual bayonet connector 102 and the female latch connector 206 from leaking during side-loads.

It will be appreciated that the male bayonet connector may include any number of ribs having various configurations. For example, each of the ribs 125(1)-125(2) may extend radially in a horizontal direction from the rib surface 126(1), and each of the ribs 125(3)-125(4) may extend radially in a horizontal direction from the rib surface 126(2). Each of the ribs 125(1)-125(4) extend in radially opposite directions from the rib surfaces 126(1)-126(2), and may be coupled to the grip 178.

The ribs 125(1)-125(4) may also have various shapes, such as a circular recess or socket. In an aspect of this disclosure, the ribs 125(1)-125(4) may extend a distance of less than 0.084-0.094 inches from the grip 178 towards the distal end of the dual shafts 122(1)-122(2).

The shafts 122(1)-122(2) may also each include an annular channel (not shown) positioned along the sealing portions 121(1)-121(2) to provide for locking of male dual bayonet connector 102 with the female latch connector 206. In alternative embodiments, the shafts 122(1)-122(2) may each define uniform circumferential surfaces that extend from the grip 178 to the distal end of the sealing portion 121(1)-121(2).

A latch member 10 may be positioned between the sealing portions 121(1)-121(2) of the shafts 122(1)-122(2) to latch the male dual lumen connector 102 to the female latch connector 206. In an aspect, the latch member 10 may extend parallel to the shafts 122(1)-122(2). The length and thickness of the latch member 10 may vary depending upon design requirements or constraints or with the relative durometer of the material used to form the connector 102. The latch member 10 may or may not be connected to the webbed portion 146 of the grip 178.

The latch member 10 includes an end portion 12, a base portion 14 positioned proximal to the end portion 12, and notches 16(1)-16(2). Each of the notches 16(1)-16(2) is positioned between the end portion 12 and the base portion 14 and provides for locking of the male dual bayonet connector 102 with the female latch connector 206. Each notch 16(1)-16(2) includes a bottom region 18(1)-18(2) that has a smaller outer diameter than an outer diameter of the end portion 12. A depth of the notches 16(1)-16(2) is defined by the difference between the diameter of the end portion 12 and the diameter of the bottom region 18(1)-18(2). In an alternative embodiment, the notches 16(1)-16(2) may form an annular ring that extends about the latch member 10.

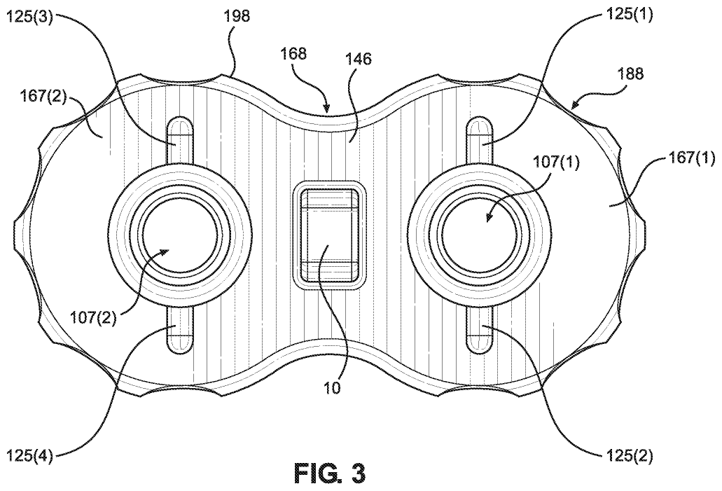

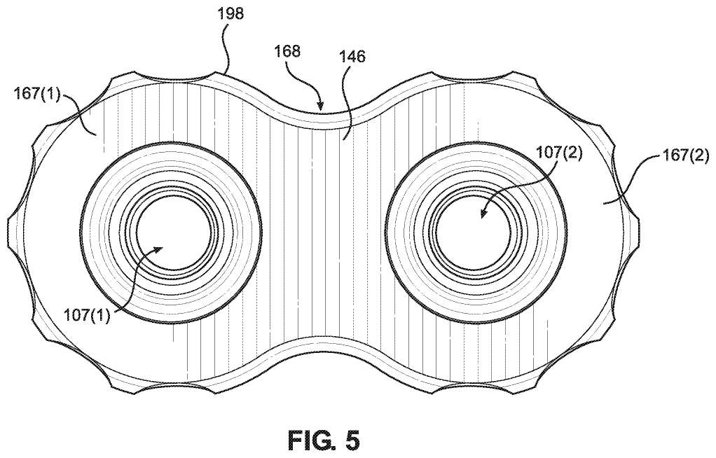

The male dual bayonet connector 102 may also include a grip 178, a portion of which may extend between the dual shafts 122(1)-122(2) to connect the shafts 122(1)-122(2) of the connector 102. In one embodiment, as best shown in FIGS. 3 and 5, the grip 178 includes two generally circular flanges 167(1)-167(2) that surround the shafts 122(1)-122(2) and that are concentric with the axes of the cylindrical lumens 107(1)-107(2). The flanges 167(1)-167(2) may be connected via a webbed portion 146 formed between the flanges 167(1)-167(2), and may have a larger outer diameter than the other portions of the shafts 122(1)-122(2) of the male dual bayonet connector 102. As such, the grip 178 may function as a stop for preventing over-insertion of the shafts 122(1)-122(2) into the female latch connector 206, and further as a guide for ensuring that the shafts 122(1)-122(2) are fully inserted into the female latch connector 206.

As illustrated in FIGS. 3 and 5, the webbed portion 146 may define a recessed area 168 between the flanges 167(1)-167(2) to allow for easy gripping of the male dual bayonet connector 102 when manipulated by a user. In addition to improving the grip of the male dual bayonet connector 102, providing a recessed area 168 in the webbed portion 146 between the flanges 167(1)-167(2) may further serve to reduce the amount of material required to manufacture the grip 178, thereby decreasing the overall cost associated with manufacturing the male dual bayonet connector 102. In alternate embodiments, there may not be a recessed area between the flanges 167(1)-167 (2) and the perimeter of the grip 178 may be in the form of an oval track with flat sidewalls.

The webbed portion 146 also provides a further benefit, in that it allows for optimal positioning of the lumens 107(1)-107(2) of the male dual bayonet connector 102 with respect to one another. In particular, the webbed portion 146 allows for positioning of the lumens 107(1)-107(2) so that the space between the central axes of the lumens 107(1)-107(2) can be maximized to allow for convenient connection and removal of both individual and webbed tubes, i.e., tubes connected with an intermediate web along their length, without modifying the tubing. In one embodiment, a distance D3 between the axis of the lumens 107(1)-107(2) may be between approximately 1.695 to 2.035 times the length D2 between the perpendicular sidewall 103(1)-103(2) and the grip 178. Additionally, a wider webbed portion 146 may position the lumens 107(1)-107(2) further apart and may help prevent tangling of the attached tubing, while a narrower webbed portion 146 would position the lumens 107(1)-107(2) closer together. A wider webbed portion 146 may alternatively allow for thicker-walled tubing to be attached to the male dual bayonet connector 102 by providing sufficient clearance for thicker tube walls. Accordingly, the width of the webbed portion 146 may be varied according to the specifications of the tubing being attached to the male dual bayonet connector 102.

Additionally, the outer edge 198 of the grip 178 may include one or more evenly-spaced indentations 188 to further facilitate gripping of the male dual bayonet connector 102 by a user. In the embodiment illustrated in FIGS. 2-6, the outer edge 198 of the grip 178 includes twelve (12) evenly-spaced indentations 188, with each ring 167(1)-167(2) including six (6) indentations 188, and a recessed webbed portion 146 extending between the rings 167(1)-167(2). However, the exact number, shape, and size of the indentations 188 are not critical so long as the grip 178 provides an enhanced gripping surface for the user. As such, in other embodiments, the number, shape, and size of the indentations 188 along the grip 178 may vary.

Another function of the grip 178 is to provide proper lead-in alignment of the male dual bayonet connector 102 with the female latch connector 206, thereby allowing for proper insertion of the male dual bayonet connector 102 into the female latch connector 206. Furthermore, the grip 178 ensures axial alignment of the shafts 122(1)-122(2) with the receiving openings 205(1)-205(2) of the female latch connector 206 during engagement, so as to allow for even distribution of the pressure applied by the sealing surface 123(1)-123(2) against the sealing member 270 to prevent leakage around the sealing surface 123(1)-123(2), as well as deformation and/or uneven wearing of the sealing member 270 over time.

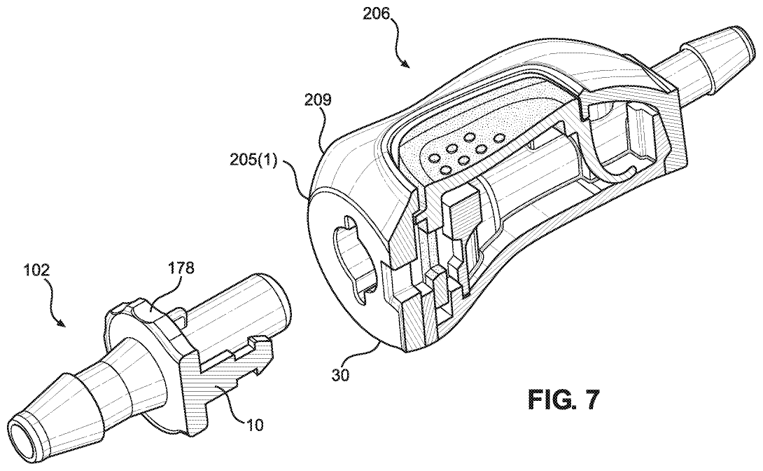

One embodiment of a female latch connector 206 that may be connected to the male dual bayonet connector 102 is illustrated in FIGS. 7-9. The female latch connector 206 may include an exterior enclosure 209 defining two openings 205(1)-205(2) for receiving the dual shafts 122(1)-122(2) of the male dual bayonet connector 102. The female latch connector 206 may further include a latch plate structure 200 defining two receiving apertures 203(1)-203(2) that are axially aligned with the exterior openings 205(1)-205(1) of the assembled female latch connector 206, so as to receive the shafts 122(1)-122(2). The latch plate structure 200 being positioned at least partially within the exterior enclosure 209. The latch plate structure 200 further defines a latch connector aperture 204 (e.g. latching slot) positioned between the two receiving apertures 203(1)-203(2) that is axially aligned with the latch member 10 of the male dual bayonet connector 102.

The female latch connector 206 may further define two cylindrical lumens 227(1)-227(2) that extend through the female latch connector 206. In one embodiment, the cylindrical lumens 227(1)-227(2) of the female latch connector 206 are positioned so that when the female latch connector 206 and the male dual lumen connector 102 are connected, the female lumens 227(1)-227(1) are axially aligned with at least a portion of the male cylindrical lumens 107(1)-107(2) to facilitate fluid flow between the connected male and female connectors 102 and 206. In other embodiments, sections of the lumens 107(1)-107(2) and 227(1)-227(2) of the male 102 or female 206 connectors may be offset with respect to one another. Additionally, the female latch connector 206 may include two tubing couplings 254(1)-254(2) that are each configured to engage a section of tubing 104(1)-104(2), as shown in FIG. 1. The tubing couplings 254(1)-254(2) of the female latch connector 206 may be similar in configuration to the male tubing couplings 156(1)-156(2)

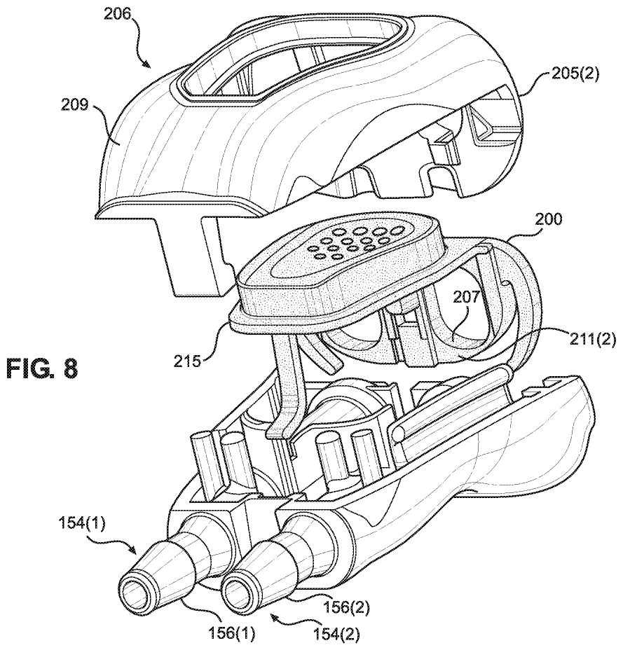

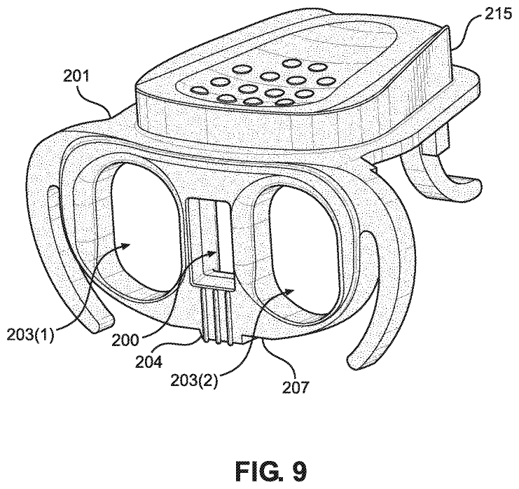

The latch plate structure 200 of the female latch connector 206 is shown in FIGS. 8 and 9. As best seen in FIG. 9, latching surface 201 may be formed along the bottom walls of the latch connector aperture 204 of the latch plate structure 200. In one embodiment, the latch plate 200 may be resiliently biased upward to lift the latching surface 201 so as to interface with the notches 16(1)-16(2) in the latch member 10 of the male dual bayonet connector 102. For example, as the latch member 10 is inserted through the latch connector aperture 204, the latch plate 200 may be biased downward to lower the latch connector aperture 204 to accommodate the outer diameter of the latch member 10. In one embodiment, end portion 12 may each be defined by a chamfered edge 20 that is angled to facilitate the insertion of the latch member 10 through the latch connector aperture 204 of the latch plate structure 200. The distal face of the latch plate 200 may define a distal latching edge 211 that may interface one of both latch shelves 22(1)-22(2) of the latch member 10 to prevent the latch member 10 from being removed from the female latch connector 206.

As the shafts 122(1)-122(2) are inserted through the receiving apertures 203(1)-203(2), the latch plate 200 may be biased downward to lower the receiving apertures 203(1)-203(2) to accommodate the outer diameter of the shafts 122(1)-122(2). In one embodiment, the receiving apertures 203(1)-203(2) may each be chamfered to facilitate the insertion of the shafts 122(1)-122(2) through the receiving apertures 203(1)-203(2) of the latch plate structure 200.

The latch surface 201 may be operably coupled to a release mechanism 215 for disengaging the latch surface 201 from the male dual bayonet connector 102. For example, as shown in FIGS. 1, 8 and 9, the release mechanism 215 may be a button that, when depressed, may lower the latch plate 200 so that the latch surface 201 may clear the notch 16(2), allowing for removal of the shafts 122(1)-122(2) from the receiving apertures 203(1)-203(2) of the female latch connector 206.

The female latch connector 206 may further include a sealing member 270 that engages the sealing surface 123(1)-123(2) of the dual shafts 122(1)-122(2) to form a fluid-tight seal between the female receiving portion 206 and the male dual bayonet connector 102. As best shown in FIG. 13, the sealing member 270 may be made from an elastomeric material that may enhance the sealing interface between the female sealing member 270 and the sealing surface 123(1)-123(2) of the male dual bayonet connector 102. The sealing member 270 may include various configurations, for example, an overmolded seal that extends the length of the sealing portion 121(1)-121(2), an overmolded seal that extends over only a portion of the length of the sealing portion 121(1)-121(2), an O-ring that has a point contact with the sealing surface 123(1)-123(2), or still other configurations.

To connect the male dual bayonet connector 102 with the female latch connector 206, the dual shafts 122(1)-122(2) may be inserted through the openings 205(1)-205(2) (shown in FIG. 1) defined in the exterior enclosure 209 of the female latch connector 206 and the receiving apertures 203(1)-203(2) defined by the latch plate structure 200, and the latch member 10 may be inserted through the latching receiving opening 30 (shown in FIG. 1) defined in the exterior enclosure 209 and the latch connector aperture 204 defined by the latch plate structure 200. Insertion of the shafts 122(1)-122(2) through the receiving apertures 203(1)-203(2) of the latch plate structure 200 and insertion of the latch member 10 through the latch connector aperture 204 of the latch plate structure 200 occurs substantially simultaneously.

With reference to FIGS. 10-12, the insertion of the latch member 10 through the latch connector aperture 204 causes the latch plate 200 to lower due to the interaction between the chamfered edge 20 of the end portion 12 and a chamfered edge 207 of the latch surface 201.

Once the shafts 122(1)-122(2) are inserted far enough so that the latch surface 201 is positioned below the notches 16(1)-16(2), the latch plate structure 200 may lift so that at least a portion of the latch surface 201 is at least partially seated within the notch 16(2), thereby preventing lateral movement of the male dual bayonet connector 102 with respect to the connected female latch connector 206.

It will be appreciated that a clearance 25 at the top of the latching slot 204 allows for the latch plate 200 to move aside and not interfere with the latch member 10. This is important to allow the latch plate 200 to move aside and then move back into the original location, where the notch 16(2) and latch plate 200 hold the male and female connectors 102 and 206 together.

The distal latching edge 211 of the latch plate 200 may interface with the latch shelf 22(2) of the notch 16(2) so as to prevent removal of the shafts 122(1)-122(2) from the female receiving portion 206. The latch shelf 22(2) resists disengagement from the latch plate 200 under longitudinal and axial loads. In one embodiment, the distal latching edge 211 of the latch plate 200 may oppose the latch shelf 22(2) defined in the latch member 10 to provide a greater axial retention force, as well as the ability to lock the male dual bayonet connector 102 with the female receiving portion 206 from the bottom of the latch member 10, as opposed to the sides of the latch member 10. This bottom locking feature further lessens the distance required for lowering the latch plate 200 to release the male dual bayonet connector 102, thereby improving the overall ergonomic design of the female latch connector 206 and minimizing the insertion force required for inserting the male dual bayonet connector 102 into the female latch connector 206.

The elongated sealing surface 123(1)-123(2) of the shafts 122(1)-122(2) may allow for positioning of the sealing mechanism 270 away from the distal end of the sealing surface 123(1)-123(2). As discussed above, this may help prevent the sealing mechanism 270 from pinching or slipping off from the distal end of the shaft 122(1)-122(2), and to sustain contact between the interior surface of the sealing mechanism 270 and the sealing surface 123(1)-123(2) to maintain a fluid-tight seal when axial forces are applied to either of the connected the male dual bayonet connector 102 or the female latch connector 206. In some embodiments, such as when the female latch connector 206 includes an O-ring or partial molded seal, the female latch connector 206 may include an additional supporting surface 216 that is positioned around the distal end of the shafts 122(1)-122(2) for providing additional axial support for the shafts 122(1)-122(2), and further preventing deformation of the sealing mechanism 270.

To remove the male dual bayonet connector 102 from the female receiving portion 206, a user may depress the release mechanism 215 to lower the latch plate 200 until the latch surfaces 201 clears the notch 16(2). Once the notch 16(2) is cleared, the male dual bayonet connector 102 may be easily disengaged from the female latch connector 206.

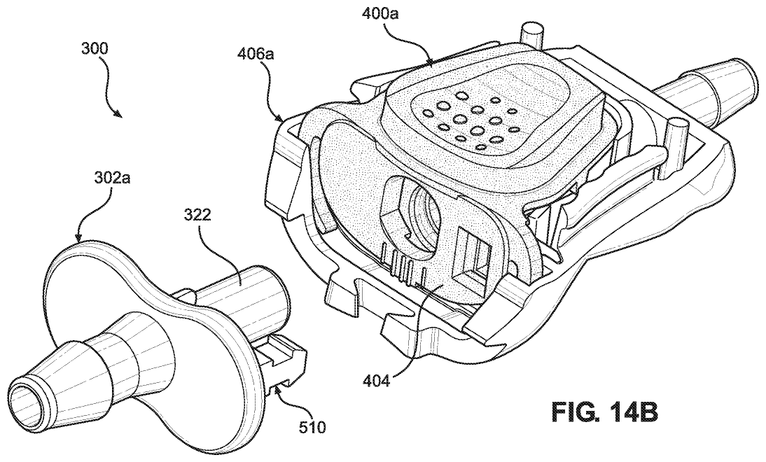

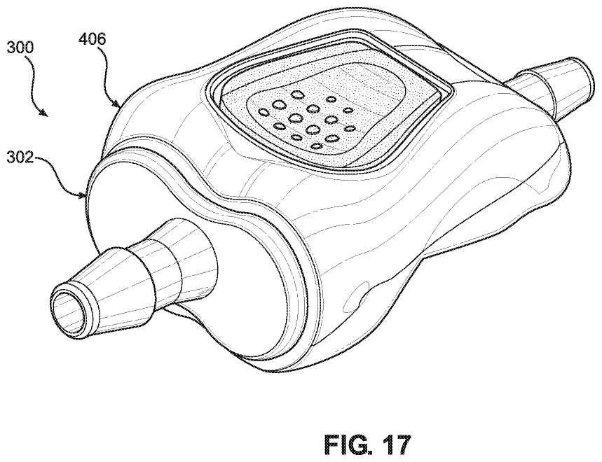

FIGS. 14-17 illustrate alternate embodiments of a releasable connection assembly 300. In the alternative embodiments, a single-lumen male bayonet connector 302 may engage with a single-lumen female receiving portion 406. Portions of the embodiments disclosed in FIGS. 14-17 are similar to aspects described above in FIGS. 1-13 and those portions function similarly to those described above.

FIG. 14A illustrates the single-lumen male bayonet connector 302a and the single-lumen female latch connector 406a, and FIG. 14B illustrates the single-lumen male bayonet connector 302a and the single-lumen female latch connector 406a with a top of the single-lumen female latch connector 406a removed. The single-lumen male bayonet connector 302a includes a latch member 510 configured substantially similarly to the latch member 10 of the male dual bayonet connector 102. The latch member 510 is positioned adjacent to a shaft 322 of the male bayonet connector 302a. The single lumen female latch connector 406a includes a latching receiving opening 430 and a latch structure 400a. The latching receiving opening 430 may be positioned offset to a center of the female latch connector 406a and is configured substantially similarly to the latching receiving opening 30 of the female latch connector 206. The latch structure 400 includes a single latch connector aperture 404 configured substantially similarly to the latch connector aperture 204 of the latch plate structure 200.

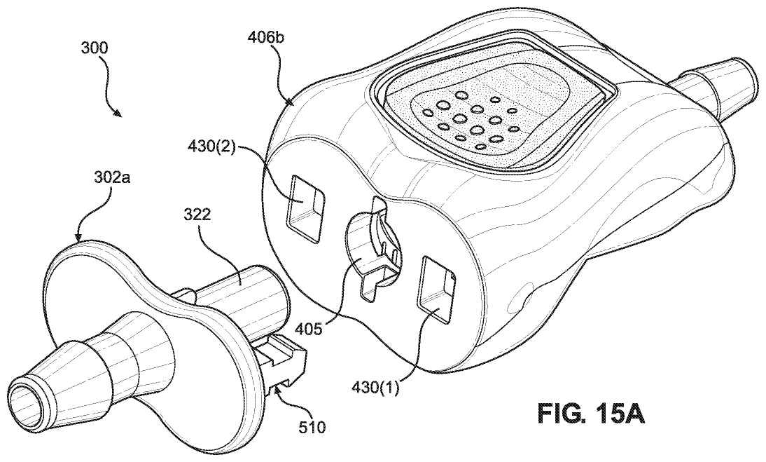

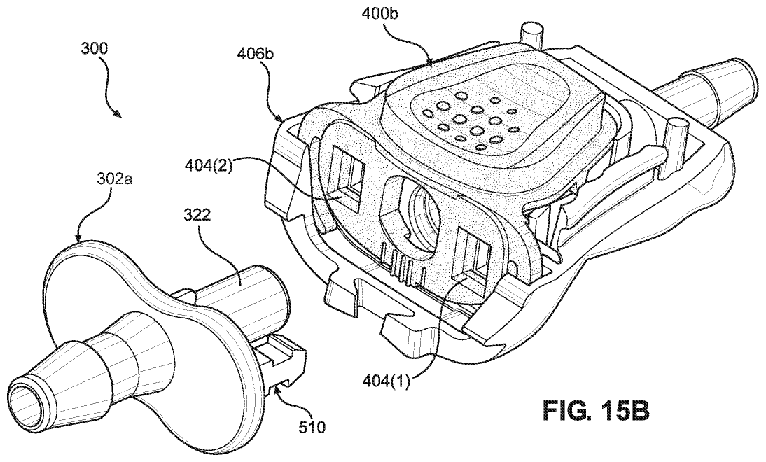

FIG. 15A illustrates the single-lumen male bayonet connector 302a and an alternative aspect of a single-lumen female latch connector 406b, and FIG. 15B illustrates the single-lumen male bayonet connector 302a and the single-lumen female latch connector 406b with a top of the single-lumen female latch connector 406b removed. The single lumen female latch connector 406b includes a first latching receiving opening 430(1), a second latching receiving opening 430(2), and a latch structure 400b. The latching receiving openings 430(1)-430(2) may be positioned on either side of a bayonet receiving opening 405 of the female latch connector 406a, and are each configured substantially similarly to the latching receiving opening 30 of the female latch connector 206. The latch structure 400b includes a first latch connector aperture 404(1) and a second latch connector aperture 404(2), each configured substantially similarly to the latch connector aperture 204 of the latch plate structure 200. Each of the first and second latching receiving openings 430(1)-430(2) and the first and second latch connector apertures 404(1)-404(2) are configured to receive the latch member 510 within. For example, the single-lumen male bayonet connector 302a may align with the single-lumen female latch connector 406b such that the latch member 510 aligns with the first latching receiving opening 430(1) and the first latch connector aperture 404(1). The male bayonet connector 302a may be rotated 180° about a single shaft 322 of the male bayonet connector 302a such that the latch member 510 aligns with the second latching receiving opening 430(2) and the second latch connector aperture 404(2).

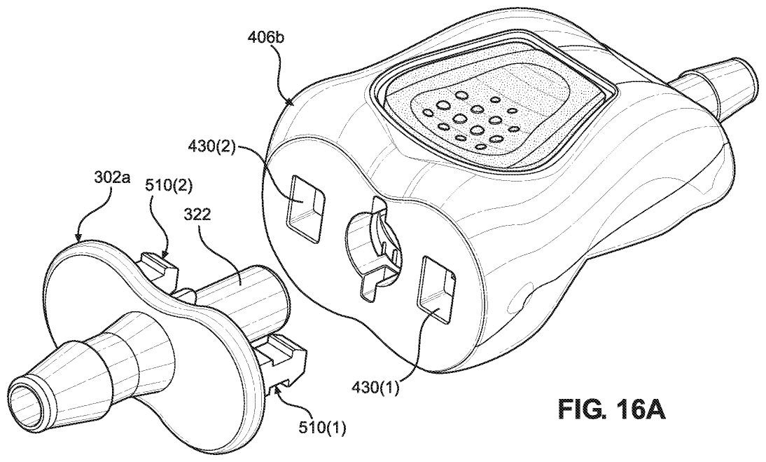

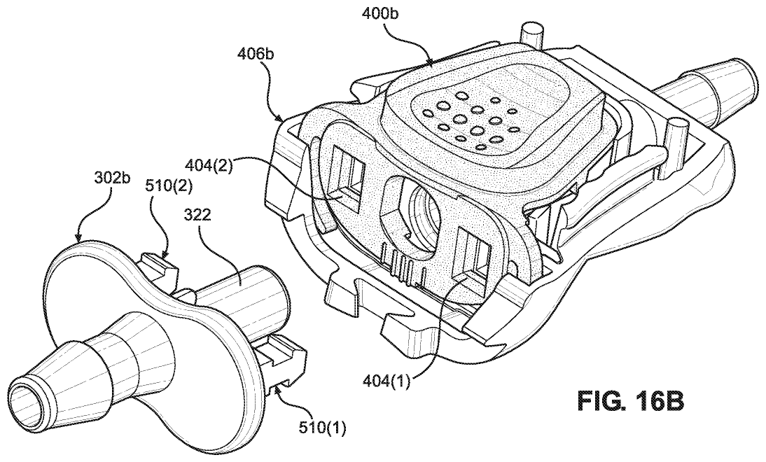

FIG. 16A illustrates an alternative aspect of a single-lumen male bayonet connector 302b and the single-lumen female latch connector 406b, and FIG. 16B illustrates the single-lumen male bayonet connector 302b and the single-lumen female latch connector 406b with a top of the single-lumen female latch connector 406b removed. The single-lumen male bayonet connector 302b includes a first latch member 510(1) and a second latch member 510(2), each configured substantially similarly to the latch member 10 of the male dual bayonet connector 102. Each of the first and second latching receiving openings 430(1)-430(2) and the first and second latch connector apertures 404(1)-404(2) are configured to receive a respective latch member 510(1)-(2) within.

It will be appreciated that the male bayonet connector may include various aspects of the grip. For example, the grip 178 may be a tie-bar.

As used herein, lumen refers not only to its definition, but also refers to an opening, aperture, or other passageway. The fluid referred to herein can be gaseous, liquid, or other state of material that is flowable through a tube (i.e., granular). In addition, while generally described above as sealed when connected together, the connector structures may be sealed or unsealed. The connection between the male dual bayonet connector and female latch connectors and their respective tube sections can be by means other than a barbed fitting, for example, but not limited to, threaded, press-fit without a barb, John Guest fitting, ferrule, and panel mount.

All directional references (e.g., upper, lower, upward, downward, left, right, leftward, rightward, top, bottom, above, below, inner, outer, vertical, horizontal, clockwise, and counterclockwise) are only used for identification purposes to aid the reader's understanding of the example of the invention, and do not create limitations, particularly as to the position, orientation, or use of the invention unless specifically set forth in the claims. Joinder references (e.g., attached, coupled, connected, joined, and the like) are to be construed broadly and may include intermediate members between a connection of elements and relative movement between elements. As such, joinder references do not necessarily infer that two elements are directly connected and in fixed relation to each other.

Although various embodiments of the invention have been described above with a certain degree of particularity, or with reference to one or more individual embodiments, those skilled in the art could make numerous alterations to the disclosed embodiments without departing from the spirit or scope of this invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A male bayonet connector, comprising:
a first shaft including a first distal end portion comprising:
a first lumen extending through the first shaft, and
a first sealing surface configured to engage a female receiving member to create a fluid-tight seal between the first lumen and a first female receiving lumen;
a second shaft including a second distal end portion comprising:
a second lumen extending through the second shaft, and
a second sealing surface configured to engage the female receiving member to create a fluid-tight seal between the second lumen and a second female receiving lumen; and
a latch member positioned between the first and second shafts, the latch member configured to couple to the female receiving member.

2. The male bayonet connector of claim 1, wherein the latch member is positioned between the first shaft and the second shaft.

3. The male bayonet connector of claim 1, wherein the latch member is configured to engage a latch plate positioned within the female receiving member.

4. The male bayonet connector of claim 1, wherein the first shaft further includes a first proximal end portion configured to engage a first section of tubing.

5. The male bayonet connector of claim 4, wherein the second shaft further includes a second proximal end portion configured to engage a second section of tubing.

6. The male bayonet connector of claim 1, further comprising a grip extending radially from the first shaft and the second shaft to define a flange around the first shaft and the second shaft.

7. The male bayonet connector of claim 6, wherein the flange defines an outer edge including a plurality of indentations for facilitating gripping of the grip.

8. The male bayonet connector of claim 6, wherein the first shaft, the second shaft, and the latch member are connected by a webbed portion of the grip.

9. The male bayonet connector of claim 8, wherein the latch member extends distally from the webbed portion of the grip.

10. The male bayonet connector of claim 6, wherein the grip comprises a tie-bar.

11. A male bayonet connector, comprising:
a shaft including a distal end portion comprising:
a lumen extending through the shaft, and
a sealing surface configured to create a fluid-tight seal between the lumen and a female receiving lumen; and
a latch member positioned adjacent to the shaft, the latch member configured to couple to a female latch structure.

12. The male bayonet connector of claim 11, further comprising a grip extending radially from the shaft and the latch member to define a flange around the shaft and the latch member.

13. The male bayonet connector of claim 12, wherein the shaft and the latch member are connected by a webbed portion of the grip.

14. The male bayonet connector of claim 13, wherein the latch member extends distally from the webbed portion of the grip.

15. A releasable connection assembly, comprising:
the male bayonet connector of claim 11; and
a female receiving member configured to couple to the male bayonet connector using the female latch structure, the female receiving member having a proximal end that defines at least two openings configured to receive at least part of the distal end portion of the shaft and the latch member.

16. The releasable connection assembly of claim 15, wherein the latch member is a first latch member, and wherein the male bayonet connector further comprises:
a second latch member positioned adjacent to the shaft, the second latch member configured to couple to the female receiving member,
wherein the proximal end of the female receiving member defines at least three openings configured to receive at least part of the distal end portion of the shaft, the first latch member, and the second latch member.

17. The releasable connection assembly of claim 16, wherein the first latch member and the second latch member are positioned on radially opposing sides of the shaft, such that the shaft is positioned between the first and second latch members.

18. The releasable connection assembly of claim 16, wherein the female receiving member comprises an exterior enclosure having the female latch structure positioned within, the female latch structure being movable relative to the exterior enclosure to release one or more of the first latch member and the second latch member.

19. A releasable connection assembly, comprising:
a male bayonet connector comprising:
a first shaft including a first distal end portion, the first distal end portion having a first sealing surface and a first lumen extending therethrough,
a second shaft including a second distal end portion, the second distal end portion having a second sealing surface and a second lumen extending therethrough, and
a latch member positioned between the first and second shafts; and
a female receiving member comprising:
a proximal end defining a first receiving lumen and a second receiving lumen configured to receive the first shaft and the second shaft within, respectively, such that a fluid-tight seal is formed between both the first shaft and the first receiving lumen, and the second shaft and the second receiving lumen,
the proximal end defining a latch receiving lumen configured to receive the latch member within, and
a latch structure configured to couple to the latch member such that motion between the male bayonet connector and the female receiving member is substantially prevented.

20. The releasable connection assembly of claim 19, wherein:
the first shaft further includes a first proximal end portion configured to engage a first section of tubing; and
the second shaft further includes a second proximal end portion configured to engage a second section of tubing.

21. The releasable connection assembly of claim 19, wherein the female receiving member further comprises an exterior enclosure with the latch structure positioned within, the female receiving member having a proximal end that defines at least three openings configured to receive at least part of the first distal end portion of the first shaft, the second distal end portion of the second shaft, and the latch member, and wherein the latch structure is movable relative to the exterior enclosure to release the latch member.

22. The releasable connection assembly of claim 19, wherein:
the first shaft further includes a first proximal end portion configured to engage a first section of tubing, and
the second shaft further includes a second proximal end portion configured to engage a second section of tubing.

23. The releasable connection assembly of claim 19, wherein:
the first shaft, the second shaft, and the latch member are connected by a webbed portion, and
the latch member extends distally from the webbed portion.
